(19) United States
(12) Patent Application Publication
(10) Pub. No.: US 2024/0006081 A1
(43) Pub. Date: Jan. 4, 2024

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Kanagawa (JP); Tomohide HIRAGAMI, Kanagawa (JP); Kenji NAGAMIYA, Kanagawa (JP); Nobuya KITAMURA, Kanagawa (JP); Yasuyuki HOSONO, Kanagawa (JP)

(21) Appl. No.: 18/455,570

(22) Filed: Aug. 24, 2023

Related U.S. Application Data
(63) Continuation of application No. PCT/JP2022/009254, filed on Mar. 3, 2022.

(30) Foreign Application Priority Data
Mar. 5, 2021 (JP) 2021-035799

(51) Int. Cl. G16H 50/70

(52) U.S. Cl. CPC G16H 50/70

(57) ABSTRACT

An information processing apparatus comprising at least one processor, wherein the processor is configured to: acquire a plurality of pieces of biological information which is biological information of a user and to which attribute information including at least one of user information regarding the user or environmental information regarding an environment at a time point when the biological information is acquired is assigned; and associate the pieces of biological information in which at least one piece of information included in the attribute information is the same.

### CROSS REFERENCE TO RELATED APPLICATIONS

This application is a continuation of International Application No. PCT/JP2022/009254, filed on Mar. 3, 2022, which claims priority from Japanese Patent Application No. 2021-035799, filed on Mar. 5, 2021. The entire disclosure of each of the above applications is incorporated herein by reference.

### BACKGROUND

### Technical Field

The present invention relates to an information processing apparatus, an information processing method, and an information processing program.

### Related Art

In the related art, a technique is known in which pieces of biological information acquired using a biological information acquisition apparatus that acquires biological information of a user are associated with each other so as to be utilized for diagnosis, health management, and the like. For example, JP2018-101255A discloses that biomedical information measured by a medical device and biological information ascertained by a biological information ascertaining device which is a non-medical device are associated with each other in time series and are utilized for health management.

In recent years, biological information of an unspecified number of users has been acquired by thermometers, a sphygmomanometer, a urine examination apparatus, and the like disposed in a town. Further, biological information such as a heart rate and arterial blood oxygen saturation is acquired by a wearable terminal worn by a user. As described above, there is a demand for a technique that enables utilization of a plurality of pieces of biological information (that is, not originally associated with each other) acquired from different users or different biological information acquisition apparatuses.

JP2018-101255A describes that biomedical information and biological information (that is, which are originally associated according to a user) acquired from the same user are associated with each other in time series, but does not describe that pieces of biological information that are not originally associated with each other are associated with each other. Therefore, in the technique described in JP2018-101255A, it is not possible to utilize a plurality of pieces of biological information that are not originally associated with each other.

### SUMMARY

The present disclosure provides an information processing apparatus, an information processing method, and an information processing program capable of utilizing biological information that is not originally associated.

According to a first aspect of the present disclosure, there is provided an information processing apparatus comprising at least one processor, in which the processor acquires a plurality of pieces of biological information which is biological information of a user and to which attribute information including at least one of user information regarding the user or environmental information regarding an environment at a time point when the biological information is acquired is assigned, and associates the pieces of biological information in which at least one piece of information included in the attribute information is the same.

According to a second aspect of the present disclosure, in the above aspect, the user information may include identification information of the user, and the processor may acquire the biological information to which identification information of a plurality of different users has been assigned, and associate the pieces of biological information to which the same identification information is assigned.

According to a third aspect of the present disclosure, in the above aspects, the user information may include action information indicating an action of the user as an acquisition source of the biological information at a time point when the biological information is acquired from the user or within a predetermined period until the time point when the biological information is acquired from the user.

According to a fourth aspect of the present disclosure, in the above aspects, the environmental information may include weather information indicating at least one of a temperature, humidity, atmospheric pressure, or weather at a time point when the biological information is acquired from the user.

According to a fifth aspect of the present disclosure, in the above aspects, the environmental information may include location information indicating a location where the biological information is acquired from the user.

According to a sixth aspect of the present disclosure, in any one of the second to fifth aspects, date and time information indicating a date and time when the biological information is acquired from the user may be assigned to the biological information, and the processor may associate the pieces of biological information with each other based on the date and time information.

According to a seventh aspect of the present disclosure, in the above aspects, the processor may acquire the plurality of pieces of biological information of different types, and may associate the pieces of biological information of different types in which at least one piece of information included in the attribute information is the same.

According to an eighth aspect of the present disclosure, in the above aspects, a type of the biological information may be at least one of a body temperature, a heart rate, an electrocardiogram, an electromyogram, blood pressure, a weight, a body fat percentage, a muscle mass, a bone density, a blood glucose level, arterial blood oxygen saturation, urine sugar, urine protein, or urine occult blood.

According to a ninth aspect of the present disclosure, in the above aspects, the biological information may be acquired by a first biological information acquisition apparatus that is publicly used by an unspecified number of users and that acquires the biological information of the unspecified number of users.

According to a tenth aspect of the present disclosure, in the ninth aspect, the first biological information acquisition apparatus may acquire the biological information in a sporadic manner.

According to an eleventh aspect of the present disclosure, in the ninth or tenth aspect, the first biological information acquisition apparatus may acquire the biological information without receiving an instruction to acquire the biological information.

According to a twelfth aspect of the present disclosure, in the above aspects, the biological information may be acquired by a second biological information acquisition apparatus that is privately used by an individual user and that acquires the biological information of the user.

According to a thirteenth aspect of the present disclosure, in the twelfth aspect, the second biological information acquisition apparatus may acquire the biological information over time.

According to a fourteenth aspect of the present disclosure, in the twelfth or thirteenth aspect, the second biological information acquisition apparatus may acquire the biological information in a case where the second biological information acquisition apparatus receives an instruction to acquire the biological information.

According to a fifteenth aspect of the present disclosure, in any one of the twelfth to fourteenth aspects, the second biological information acquisition apparatus may be wearable.

According to a sixteenth aspect of the present disclosure, there is provided an information processing method executed by a computer, the method comprising: acquiring a plurality of pieces of biological information which is biological information of a user and to which attribute information including at least one of user information regarding the user or environmental information regarding an environment at a time point when the biological information is acquired is assigned; and associating the pieces of biological information in which at least one piece of information included in the attribute information is the same.

According to a seventeenth aspect of the present disclosure, there is provided an information processing program for causing a computer to execute a process comprising: acquiring a plurality of pieces of biological information which is biological information of a user and to which attribute information including at least one of user information regarding the user or environmental information regarding an environment at a time point when the biological information is acquired is assigned; and associating the pieces of biological information in which at least one piece of information included in the attribute information is the same.

According to the above aspects, the information processing apparatus, the information processing method, and the information processing program of the present disclosure enable utilization of biological information that is not originally associated.

### BRIEF DESCRIPTION OF THE DRAWINGS

FIG. 1 is a schematic configuration diagram of an information processing system.

FIG. 2 is a block diagram illustrating an example of a hardware configuration of an information processing apparatus.

FIG. 3 is a block diagram illustrating an example of a functional configuration of the information processing apparatus.

FIG. 4 is a diagram illustrating an example of biological information and attribute information.

FIG. 5 is a diagram illustrating an example of a result in which pieces of biological information including the same identification information of a user are associated with each other.

FIG. 6 is a diagram illustrating an example of a result in which pieces of biological information including the same action information are associated with each other.

FIG. 7 is a diagram illustrating an example of a screen displayed on a display.

FIG. 8 is a flowchart illustrating an example of information processing.

### DETAILED DESCRIPTION

Hereinafter, exemplary embodiments for implementing the technology of the present disclosure will be described in detail with reference to the drawings.

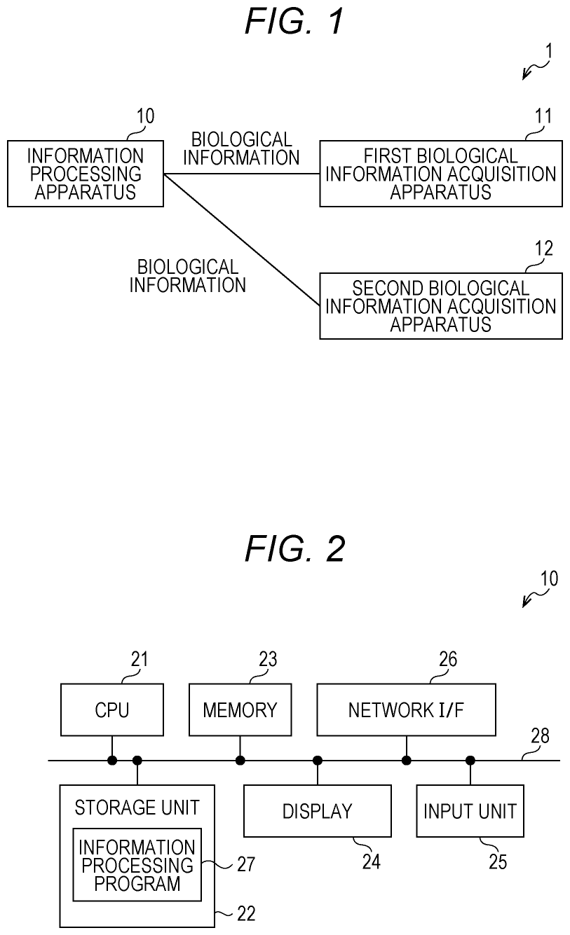

An example of a configuration of an information processing system 1 according to the present exemplary embodiment will be described with reference to FIG. 1. As illustrated in FIG. 1, the information processing system 1 includes an information processing apparatus 10, at least one first biological information acquisition apparatus 11, and at least one second biological information acquisition apparatus 12. Although FIG. 1 illustrates one first biological information acquisition apparatus 11 and one second biological information acquisition apparatus 12, the information processing system 1 may include a plurality of first biological information acquisition apparatuses 11 and a plurality of second biological information acquisition apparatuses 12. The information processing apparatus 10 and the first biological information acquisition apparatus 11 can communicate with each other via wired or wireless communication. The information processing apparatus 10 and the second biological information acquisition apparatus 12 can communicate with each other via wired or wireless communication.

Hereinafter, in a case where the first biological information acquisition apparatus 11 and the second biological information acquisition apparatus are not distinguished from each other, the first biological information acquisition apparatus 11 and the second biological information acquisition apparatus 12 are simply referred to as “biological information acquisition apparatus”. The biological information acquisition apparatus has a function of acquiring biological information of a user. The biological information is, for example, information indicating at least one of a body temperature, a heart rate, an electrocardiogram, an electromyogram, blood pressure, a weight, a body fat percentage, a muscle mass, a bone density, a blood glucose level, arterial blood oxygen saturation, urine sugar, urine protein, or urine occult blood (details will be described later).

In the present exemplary embodiment, the first biological information acquisition apparatus 11 is an apparatus that is publicly used by an unspecified number of users and that acquires biological information of the unspecified number of users. The term “publicly” means that the first biological information acquisition apparatus 11 is installed in a place where people gather (for example, a hospital, a pharmacy, a department store, a shopping mall, a convenience store, a station, an airport, a company, a public building, an elderly facility, a hot spring facility, or the like), and can be used by anyone. As the first biological information acquisition apparatus 11, for example, a thermometer provided at an entrance/exit of a store or the like, a urine examination apparatus provided in a public restroom, or the like can be used. In a case where a plurality of first biological information acquisition apparatuses 11 are provided, the plurality of first biological information acquisition apparatuses 11 may be installed at different locations.

In addition, the first biological information acquisition apparatus 11 acquires biological information in a sporadic manner without receiving an instruction to acquire biological information from a user. Specifically, the first biological information acquisition apparatus 11 automatically acquires biological information of an unspecified number of users who have entered a range in which biological information can be measured. That is, from the viewpoint of the user, the biological information of the user is passively acquired by the first biological information acquisition apparatus 11.

On the other hand, in the present exemplary embodiment, the second biological information acquisition apparatus 12 is privately used by an individual user and acquires biological information of the user. The term “privately” means that only the user who is the owner of the second biological information acquisition apparatus 12 can use the second biological information acquisition apparatus 12. As the second biological information acquisition apparatus 12, for example, a wearable terminal such as a smartwatch, a blood glucose self-measuring device, or the like can be used.

The second biological information acquisition apparatus 12 acquires biological information in a case where the second biological information acquisition apparatus 12 receives an instruction to acquire the biological information from the user. For example, the user gives an instruction to measure a heart rate to a smartwatch worn on a wrist of the user. That is, from the viewpoint of the user, the second biological information acquisition apparatus 12 actively acquires the biological information of the user.

Note that the second biological information acquisition apparatus 12 may acquire biological information over time. For example, the heart rate may be acquired over time by the smartwatch. In this case, the user instructs the smartwatch worn on the wrist to start the measurement of the heart rate over time.

An example of a hardware configuration of the information processing apparatus 10 according to the present exemplary embodiment will be described with reference to FIG. 2. As illustrated in FIG. 2, the information processing apparatus 10 includes a central processing unit (CPU) 21, a nonvolatile storage unit 22, and a memory 23 as a temporary storage area. In addition, the information processing apparatus 10 includes a display 24 such as a liquid crystal display, an input unit 25 such as a keyboard, a mouse, and a button, and a network interface (I/F) 26 that performs wired or wireless communication with the biological information acquisition apparatus and an external network (not illustrated). The CPU 21, the storage unit 22, the memory 23, the display 24, the input unit 25, and the network OF 26 are connected to each other via a bus 28, such as a system bus and a control bus, such that the CPU 21, the storage unit 22, the memory 23, the display 24, the input unit 25, and the network OF 26 can transmit and receive various kinds of information to and from each other. As the information processing apparatus 10, for example, a smartphone, a wearable terminal, a personal computer, a server computer, or the like can be used.

The storage unit 22 is implemented by, for example, a storage medium such as a hard disk drive (HDD), a solid-state drive (SSD), or a flash memory. An information processing program 27 is stored in the storage unit 22 in the information processing apparatus 10. The CPU 21 reads the information processing program 27 from the storage unit 22, loads the information processing program 27 into the memory 23, and executes the loaded information processing program 27. The CPU 21 is an example of a processor according to the present disclosure.

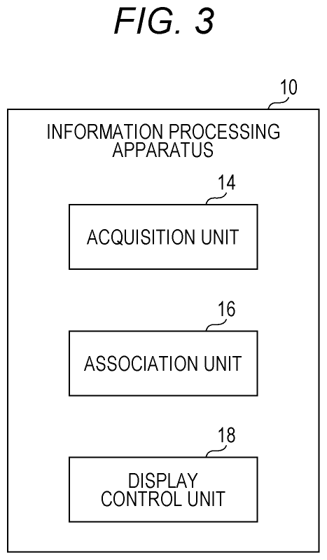

Next, an example of a functional configuration of the information processing apparatus 10 according to the present exemplary embodiment will be described with reference to FIG. 3. As illustrated in FIG. 3, the information processing apparatus 10 includes an acquisition unit 14, an association unit 16, and a display control unit 18. The CPU 21 functions as the acquisition unit 14, the association unit 16, and the display control unit 18 by executing the information processing program 27.

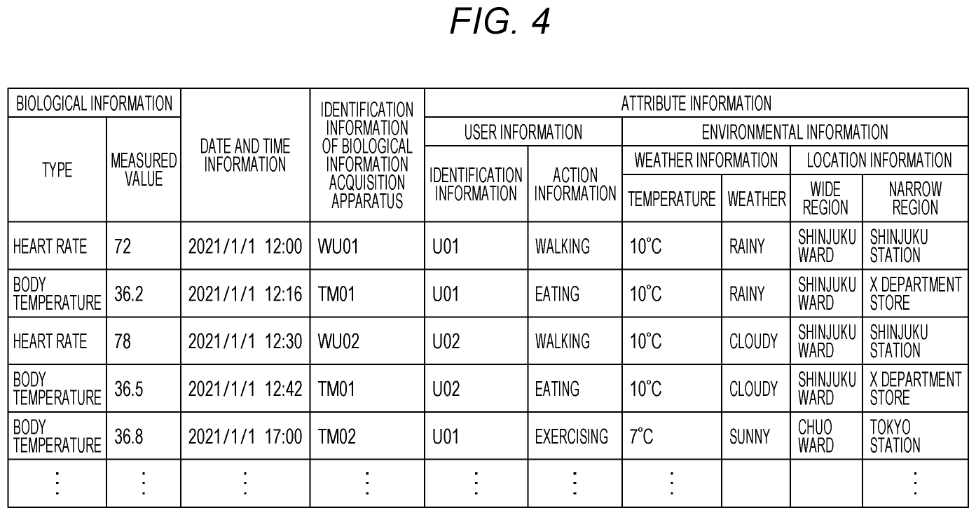

The acquisition unit 14 acquires a plurality of pieces of biological information of the user from the biological information acquisition apparatus. FIG. 4 illustrates an example of the biological information according to the present exemplary embodiment. As illustrated in FIG. 4, the biological information includes types of the biological information and measurement results of the biological information, and the acquisition unit 14 acquires different types of the biological information. The measurement results may be represented by measured values as illustrated in FIG. 4, or may be represented by determination results such as negative or positive or good or poor based on the measured values.

Date and time information indicating a date and time when the biological information is acquired from the user and identification information of the biological information acquisition apparatus as an acquisition source are assigned to the biological information. The date and time information and the identification information of the biological information acquisition apparatus are assigned to the biological information by the biological information acquisition apparatus and transmitted to the information processing apparatus 10.

Further, attribute information including at least one of user information regarding the user as an acquisition source or environmental information regarding an environment at a time point when the biological information is acquired is assigned to the biological information. The attribute information may be transmitted to the information processing apparatus 10 after being assigned to the biological information by the biological information acquisition apparatus, or may be assigned after the information processing apparatus 10 acquires the biological information.

The user information includes identification information of the user and action information indicating an action of the user as an acquisition source at the time point when the biological information is acquired from the user. The “identification information of the user” is information that can identify the user as the acquisition source of the biological information and is unique to each user. That is, the acquisition unit 14 acquires, from the biological information acquisition apparatus, biological information to which identification information of a plurality of different users has been assigned.

For example, in a case where the first biological information acquisition apparatus 11 acquires biological information, the first biological information acquisition apparatus 11 may detect a terminal device (for example, a smartphone, a wearable terminal, or the like) owned by an individual user as an acquisition source of the biological information and may assign identification information of the detected terminal device to the biological information as identification information of the user. The detection of the terminal device may be performed by using known short-range wireless communication such as Bluetooth (registered trademark), a Bluetooth Low Energy (BLE) beacon, and a radio frequency identifier (RFID). In addition, for example, in a case where the second biological information acquisition apparatus 12 acquires biological information, the second biological information acquisition apparatus 12 may ascertain identification information of the user who is the owner of the second biological information acquisition apparatus 12 in advance, and assign the ascertained identification information of the user to the biological information at the time of acquiring the biological information.

Examples of the “action information” include walking, exercising, resting, sleeping, waking up, eating, excreting, working, and engaging in leisure activities. For example, in a case where a smart watch including an acceleration sensor is used as the biological information acquisition apparatus, the biological information acquisition apparatus may use the acceleration sensor to detect which action of “walking”, “exercising”, or “resting” the user is performing, and may assign action information indicating the detected action to the biological information. For example, in a case where the biological information acquisition apparatus is installed in a restaurant, the biological information acquisition apparatus may assign action information of “eating” to the biological information. Further, for example, after the information processing apparatus 10 acquires the biological information, the information processing apparatus 10 may receive an input of the action information by the user via the input unit 25, and may assign the received action information to the biological information.

Note that the action information may be information indicating an action of the user as the acquisition source within a predetermined period until the time point when the biological information is acquired from the user. For example, even in a case where the biological information is acquired after an action, for example, after eating and exercise or the like (that is, in a case where the user is not doing anything at the time of acquisition of the biological information), action information of “eating” or “exercising” may be assigned.

The environmental information includes weather information indicating at least one of a temperature, humidity, atmospheric pressure, or weather at the time point when the biological information is acquired from the user. For example, the biological information acquisition apparatus may include a sensor that measures the temperature, and the temperature measured by the sensor may be assigned to the biological information as the weather information. Further, for example, the information processing apparatus 10 may access a server in which weather information for each time at each location is accumulated to acquire the weather information at the time of acquisition of the biological information based on the date and time information and location information (details will be described below) and may assign the acquired weather information to the biological information.

The environmental information includes the location information indicating the location where the biological information is acquired from the user. Examples of the “location information” include prefectures, municipalities, facilities, as well as structures within facilities, such as entrances, exits, and restrooms. Further, as illustrated in FIG. 4, the location information may be subdivided in stages, such as location information of a “wide region” corresponding to a municipality and location information of a “narrow region” corresponding to a facility. For example, in a case where a smartwatch having a global positioning system (GPS) function is used as the biological information acquisition apparatus, location information indicating a location detected by the biological information acquisition apparatus using the GPS function may be assigned to the biological information. In addition, for example, the information processing apparatus 10 may access a table in which the identification information of the biological information acquisition apparatus and the installation location of the biological information acquisition apparatus are stored in association with each other to acquire the location information on the basis of the identification information of the biological information acquisition apparatus and assign the location information to the biological information.

The association unit 16 associates pieces of biological information in which at least one piece of information included in the attribute information is the same. The association unit 16 may associate pieces of biological information of different types with each other as long as at least one piece of information included in the attribute information. For example, the association unit 16 may associate different types of biological information, such as a body temperature and a heart rate, or the body temperature and urine protein. The combination of different types of biological information may be a combination that is not used in a normal disease examination.

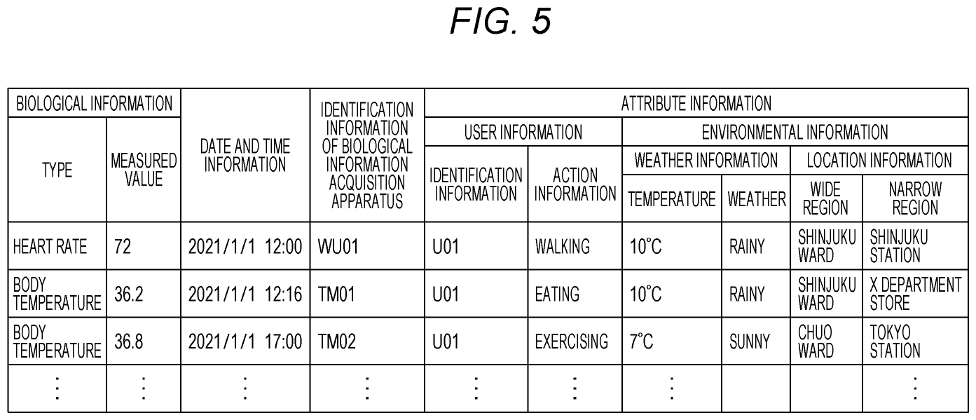

As an example, FIG. 5 illustrates an example in which pieces of biological information to which “U01” is assigned as identification information of a user are associated with each other. Since the association unit 16 associates the pieces of biological information having the same identification information of the user with each other, a plurality of pieces of biological information of a certain user can be comprehensively used for diagnosis, health management, and the like.

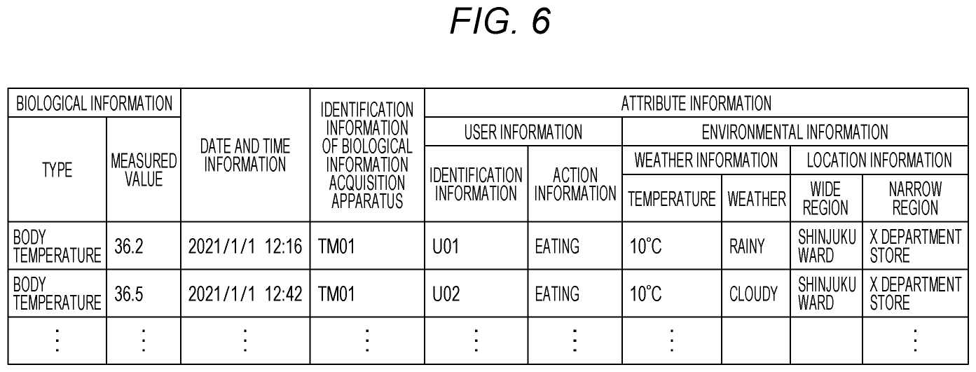

In addition, FIG. 6 illustrates an example in which pieces of biological information to which “eating” is assigned as action information are associated with each other. In a case where the association unit 16 associates pieces of biological information having the same action information with each other, the biological information can be used for analysis such as ascertaining a general tendency of biological information of a plurality of users at a time point when a certain action is performed (or within a predetermined period before the action is performed). For example, by comparing biological information for each piece of action information, it may be possible to ascertain a tendency such as deterioration (or improvement) of a symptom specifically indicated by biological information in a certain action.

In addition, the association unit 16 may associate pieces of biological information to which the same weather information and/or the same location information is assigned. For example, when pieces of biological information acquired in an area in which COVID-19 is prevalent are associated with each other and analyzed, and a result of the analysis is used, the epidemic situation of COVID-19 can be estimated from the tendency of biological information of a user in another region in some cases.

Further, the association unit 16 may associate the pieces of biological information in a stepwise manner based on the attribute information. For example, the association unit 16 may associate pieces of biological information of one user with each other on the basis of the identification information of the user and may further associate some pieces of the biological information with each other on the basis of the action information.

The association unit 16 may associate the pieces of biological information with each other on the basis of the attribute information and then further associate the pieces of biological information with each other on the basis of the date and time information. For example, the association unit 16 may associate pieces of biological information of one user with each other on the basis of the identification information of the user and then further associate some pieces of biological information to which date and time information indicating a time within a predetermined period has been assigned. For example, the “predetermined period” may be set to a time zone such as morning, afternoon, or evening, may be set to a date such as one day, one week, one month, or one year, or may be set to a season such as spring, summer, autumn, or winter, or a rainy season or a dry season.

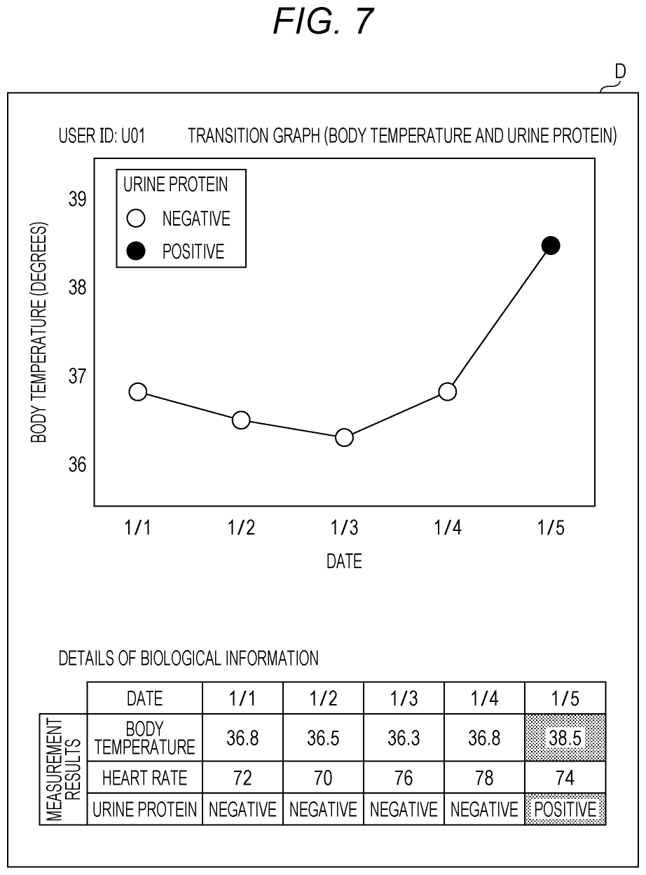

The display control unit 18 performs various kinds of guidance to the user by performing control to display a screen D on the display 24. FIG. 7 illustrates an example of the screen D displayed on the display 24 by the display control unit 18. FIG. 7 illustrates an example in which the association unit 16 associates pieces of biological information of “body temperature”, “heart rate”, and “urine protein” to which “U01” is assigned as the identification information of the user, and further associates the pieces of biological information for each day based on date and time information. As illustrated in FIG. 7, the display control unit 18 displays the maximum body temperature for each date by a line graph, and displays the determination result of urine protein for each date by changing a color of a plot. As described above, the display control unit 18 may process and display the associated pieces of biological information in an easy-to-see format using a graph, color coding, or the like.

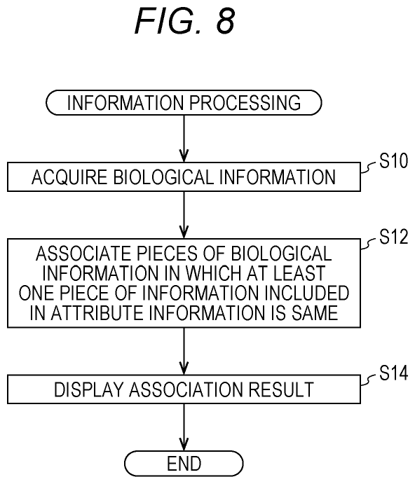

Next, an operation of the information processing apparatus 10 according to the present exemplary embodiment will be described with reference to FIG. 8. In the information processing apparatus 10, the CPU 21 executes the information processing program 27 to execute information processing illustrated in FIG. 8. The information processing is executed, for example, in a case where an instruction to start the execution is input by the user via the input unit 25.

In step S10, the acquisition unit 14 acquires, from the biological information acquisition apparatus, a plurality of pieces of biological information to which the attribute information including at least one of the user information or the environmental information is assigned. In step S12, the association unit 16 associates pieces of biological information in which at least one piece of information included in the attribute information is the same among the plurality of pieces of biological information acquired in step S10. In step S14, the display control unit 18 displays the pieces of biological information associated in step S12 on the display 24, and ends the information processing.

Although the case where the attribute information has already been assigned to the biological information in step S10 in the information processing illustrated in FIG. 8 (that is, the attribute information has already been assigned to the biological information by the biological information acquisition apparatus) is described, but the present disclosure is not limited thereto. For example, the acquisition unit 14 may acquire the biological information to which the attribute information is not assigned from the biological information acquisition apparatus in step S10, and the acquisition unit 14 may assign the attribute information to the biological information after step S10.

As described above, the information processing apparatus 10 includes at least one processor, and the processor acquires a plurality of pieces of biological information which is biological information of a user and to which attribute information including at least one of user information regarding the user or environmental information regarding an environment at a time point when the biological information is acquired is assigned, and associates pieces of biological information in which at least one piece of information included in the attribute information is the same. That is, since the pieces of biological information can be associated with each other on the basis of the user information and/or the environmental information assigned to the biological information, the biological information which is not originally associated can be used for diagnosis, health management, analysis, and the like.

Although the exemplary embodiment describes the case where the acquisition unit 14 acquires the biological information of the plurality of different users from the biological information acquisition apparatus, the present disclosure is not limited thereto. For example, the acquisition unit 14 may acquire only a plurality of pieces of biological information of one certain user from the biological information acquisition apparatus.

In addition, although the exemplary embodiment describes, in detail, the form in which both the user information and the environmental information are assigned as the attribute information to the biological information, the present disclosure is not limited thereto. The attribute information including at least one of the user information or the environmental information may be assigned to the biological information, and any one of the user information and the environmental information may not be assigned to the biological information. Furthermore, both the user information and the environmental information may be assigned to some of the plurality of pieces of biological information, and only one of the user information and the environmental information may be assigned to some of the plurality of pieces of biological information. For example, even in a case where the user information is not assigned to the biological information (that is, the individual as the acquisition source of the biological information cannot be specified), the association unit 16 associates pieces of biological information having the same environmental information with each other, and thus, analysis or the like of the biological information according to the environmental information can be performed.

In the exemplary embodiment, for example, the following various processors can be used as a hardware structure of the processing units that perform the various processes, such as the acquisition unit 14, the association unit 16, and the display control unit 18. As described above, in addition to the CPU which is a general-purpose processor that executes software (program) to function as the various processing units, examples of the various processors include a programmable logic device (PLD) which is a processor having a circuit configuration which is changeable after a field programmable gate array (FPGA) or the like is manufactured, and a dedicated electric circuit which is a processor having a circuit configuration designed as a dedicated circuit in order to perform specific processing, such as an application specific integrated circuit (ASIC).

Each of the processing units may be configured by one of these various processors or may be configured by a combination of two or more processors of the same type or different types (for example, a combination of a plurality of FPGAs or a combination of a CPU and an FPGA). In addition, a plurality of processing units may be configured by one processor.

As an example in which a plurality of processing units are configured by one processor, first, as represented by a computer such as a client and a server, there is a form in which one processor is configured by a combination of one or more CPUs and software and this processor functions as the plurality of processing units. Second, as represented by a system on chip (SoC) or the like, there is a form in which a processor that implements the functions of the entire system including the plurality of processing units with one integrated circuit (IC) chip is used. As described above, the various processing units are configured using one or more of the various processors as the hardware structure.

Further, more specifically, an electric circuit (circuitry) in which circuit elements such as semiconductor elements are combined can be used as the hardware structure of the various processors.

Further, although the above-described exemplary embodiment describes the configuration in which the information processing program 27 is stored (installed) in the storage unit 22 in advance, the present disclosure is not limited thereto. The information processing program 27 may be provided in a form of being recorded on a recording medium such as a compact disc read-only memory (CD-ROM), a digital versatile disc read-only memory (DVD-ROM), or a Universal Serial Bus (USB) memory. In addition, the information processing program 27 may be downloaded from an external device via a network.

Furthermore, the technology of the present disclosure extends to a storage medium that stores the information processing program in a non-transitory manner, in addition to the information processing program.

In the technique of the present disclosure, the above-described exemplary embodiments can be appropriately combined. The details of the above descriptions and illustrations are detailed descriptions of the portions according to the technology of the present disclosure and are merely examples of the technology of the present disclosure. For example, the above description of the configurations, the functions, the operations, and the effects is an example of the configurations, functions, operations, and effects of the portions according to the technology of the present disclosure. Accordingly, it goes without saying that unnecessary portions may be removed, new elements may be added, or replacement may be made with respect to the details of the above descriptions and illustrations without departing from the scope of the technology of the present disclosure.

The disclosure of JP2021-035799 filed on Mar. 5, 2021 is incorporated herein by reference in its entirety. All the documents, patent applications, and technical standards described in this specification are herein incorporated by reference to the same extent as if each document, patent application, or technical standard was specifically and individually indicated to be incorporated by reference.

What is claimed is:

1. An information processing apparatus comprising at least one processor, wherein the processor is configured to:
acquire a plurality of pieces of biological information which is biological information of a user and to which attribute information including at least one of user information regarding the user or environmental information regarding an environment at a time point when the biological information is acquired is assigned; and
associate the pieces of biological information in which at least one piece of information included in the attribute information is the same.

2. The information processing apparatus according to claim 1, wherein:
the user information includes identification information of the user, and
the processor is configured to:
acquire the biological information to which identification information of a plurality of different users has been assigned; and
associate the pieces of biological information to which the same identification information is assigned.

3. The information processing apparatus according to claim 1, wherein the user information includes action information indicating an action of the user as an acquisition source of the biological information at a time point when the biological information is acquired from the user or within a predetermined period until the time point when the biological information is acquired from the user.

4. The information processing apparatus according to claim 1, wherein the environmental information includes weather information indicating at least one of a temperature, humidity, atmospheric pressure, or weather at a time point when the biological information is acquired from the user.

5. The information processing apparatus according to claim 1, wherein the environmental information includes location information indicating a location where the biological information is acquired from the user.

6. The information processing apparatus according to claim 2, wherein:
date and time information indicating a date and time when the biological information is acquired from the user is assigned to the biological information, and
the processor is configured to associate the pieces of biological information with each other based on the date and time information.

7. The information processing apparatus according to claim 1, wherein the processor is configured to:
acquire the plurality of pieces of biological information of different types; and
associate the pieces of biological information of different types in which at least one piece of information included in the attribute information is the same.

8. The information processing apparatus according to claim 1, wherein a type of the biological information is at least one of a body temperature, a heart rate, an electrocardiogram, an electromyogram, blood pressure, a weight, a body fat percentage, a muscle mass, a bone density, a blood glucose level, arterial blood oxygen saturation, urine sugar, urine protein, or urine occult blood.

9. The information processing apparatus according to claim 1, wherein the biological information is acquired by a first biological information acquisition apparatus that is publicly used by an unspecified number of users and that acquires the biological information of the unspecified number of users.

10. The information processing apparatus according to claim 9, wherein the first biological information acquisition apparatus acquires the biological information in a sporadic manner.

11. The information processing apparatus according to claim 9, wherein the first biological information acquisition apparatus acquires the biological information without receiving an instruction to acquire the biological information.

12. The information processing apparatus according to claim 1, wherein the biological information is acquired by a second biological information acquisition apparatus that is privately used by an individual user and that acquires the biological information of the user.

13. The information processing apparatus according to claim 12, wherein the second biological information acquisition apparatus acquires the biological information over time.

14. The information processing apparatus according to claim 12, wherein the second biological information acquisition apparatus acquires the biological information in a case where the second biological information acquisition apparatus receives an instruction to acquire the biological information.

15. The information processing apparatus according to claim 12, wherein the second biological information acquisition apparatus is wearable.

16. An information processing method executed by a computer, the method comprising:
acquiring a plurality of pieces of biological information which is biological information of a user and to which attribute information including at least one of user information regarding the user or environmental information regarding an environment at a time point when the biological information is acquired is assigned; and
associating the pieces of biological information in which at least one piece of information included in the attribute information is the same.

17. A non-transitory computer-readable storage medium storing an information processing program for causing a computer to execute a process comprising:
acquiring a plurality of pieces of biological information which is biological information of a user and to which attribute information including at least one of user information regarding the user or environmental information regarding an environment at a time point when the biological information is acquired is assigned; and
associating the pieces of biological information in which at least one piece of information included in the attribute information is the same.
